(19) United States
(12) Patent Application Publication
(10) Pub. No.: US 2010/0076013 A1
(43) Pub. Date: Mar. 25, 2010

(54) Methods of Treatment

(75) Inventors: Leigh Zawel, Cambridge, MA (US); Yao Yao, Cambridge, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: Novartis AG

(21) Appl. No.: 12/516,511

(22) PCT Filed: Nov. 26, 2007
(86) PCT No.: PCT/US07/85486

§ 371 Date: May 27, 2009

Related U.S. Application Data
(60) Provisional application No. 60/867,450, filed on Nov. 28, 2006.

(51) Int. Cl. A61K 31/437; A61K 31/427; A61P 35/02

(52) U.S. Cl. 514/300; 514/365

(57) ABSTRACT

The present invention relates to methods of treating hematological malignancies, including acute myeloid leukemia (AML), comprising the use of compounds that inhibit the binding of the Smac protein to IAPs (“IAP inhibitor”). The present invention also relates to the use of IAP inhibitors for the preparation of a medicament to treat hematological malignancies, including AML.

### FIELD OF THE INVENTION

The present invention relates to methods of treating hematological malignancies, including acute myeloid leukemia (AML), comprising the use of compounds that inhibit the binding of the Smac protein to IAPs (“IAP inhibitor”).

The present invention also relates to the use of IAP inhibitors for the preparation of a medicament to treat hematological malignancies, including AML.

### BACKGROUND OF THE INVENTION

AML is a hematologic malignancy characterized by a block in cellular differentiation and aberrant growth of myeloid precursor cells. Approximately 30% of AML patients, and a portion of acute lymphoblastic leukemia (ALL) patients, express a mutated form of the class III receptor tyrosine kinase, FLT3 (Fms-Like Tyrosine kinase-3; STK-1, human Stem Cell Tyrosine Kinase-1; or FLK-2, Fetal Liver Kinase-2). See Rosnet and Birnbaum (1993) and Stirewalt and Radich (2003). Constitutively activated FLT3 occurs most often as internal tandem duplications within the juxtamembrane domain [see Nakao et al. (1996)], and is observed in approximately 20-25% of AML patients, but in less than 5% of patients with myelodysplastic syndrome (MDS). See Nakao et al. (1996); Horiike et al. (1997); Kiyoi et al. (1998); Kondo et al. (1999); Kiyoi et al. (1999) and Rombouts et al. (2000). The transplantation of murine bone marrow cells infected with a retrovirus expressing a FLT3-ITD mutant has been shown to lead to the development of a rapidly lethal myeloproliferative disease in mice. See Kelly et al. (2002). Gain-of-function FLT3 occurs less often as point mutations in the activation loop (in approximately 7% of AML cases), and is often characterized by an asparagine (Asp) residue at position 835. See Yamamoto et al. (2001). Occurring less frequently are additional point mutations in the kinase domain, including N841I [see Jiang et al. (2004)] and Y842C [see Kindler et al. (2005)]. There is a need to develop small molecules for the treatment of acute leukemia patients.

### SUMMARY OF THE INVENTION

It has been found that members of the IAP protein family play a role in mediating apoptosis and these proteins are a viable target in leukemia, as they have been found to be variably expressed in acute leukemias, and are associated with chemosensitivity, chemoresistance, disease progression, remission, and patient survival.

The present invention relates to a method of treating a warm-blooded animal, especially a human, having leukemia, especially AML, in particular, AML which is resistant to conventional chemotherapy, comprising administering to said animal a therapeutically effective amount of an IAP inhibitor; useful in AML treatment.

In another embodiment, the present invention relates to the use of IAP inhibitors in the preparation of a medicament for the treatment of hematological malignancies, including AML.

### DETAILED DESCRIPTION OF THE DRAWINGS

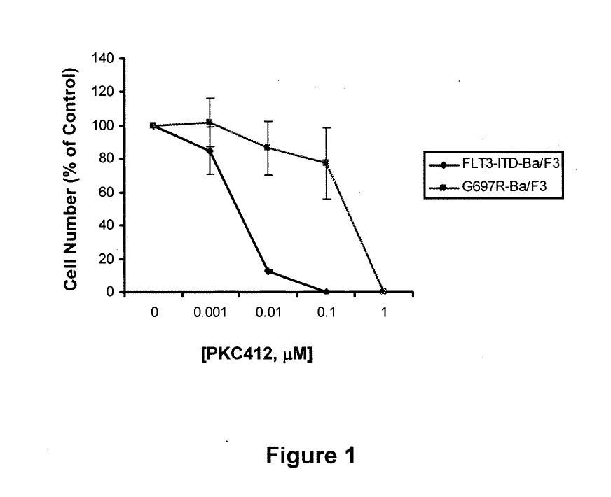

FIG. 1 illustrates two-day treatment of N-[1-cyclohexyl-2-oxo-2-(6-phenethyl-octahydro-pyrrolo[2,3-c]pyridin-1-yl-ethyl]-2-methylamino-propionamide on PKC412-sensitive FLT3-ITD-Ba/F3 and PKC412-resistant G697R-Ba/F3 with PKC412 (n=2).

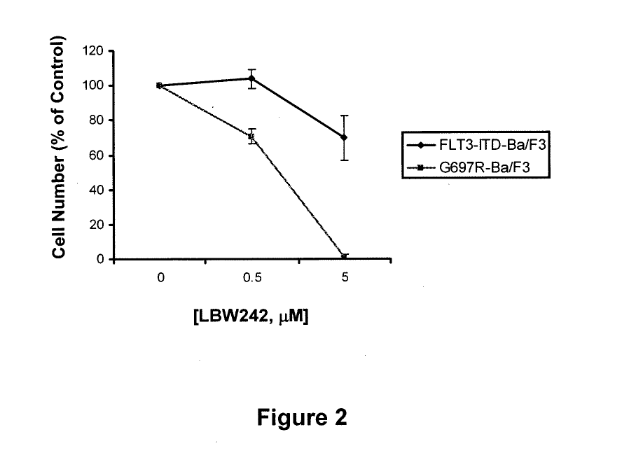

FIG. 2 illustrates three-day treatment of N-[1-cyclohexyl-2-oxo-2-(6-phenethyl-octahydro-pyrrolo[2,3-c]pyridin-1-yl-ethyl]-2-methylamino-propionamide on PKC412-sensitive FLT3-ITD-Ba/F3 and PKC412-resistant G697R-Ba/F3 with LBW242 (n=2).

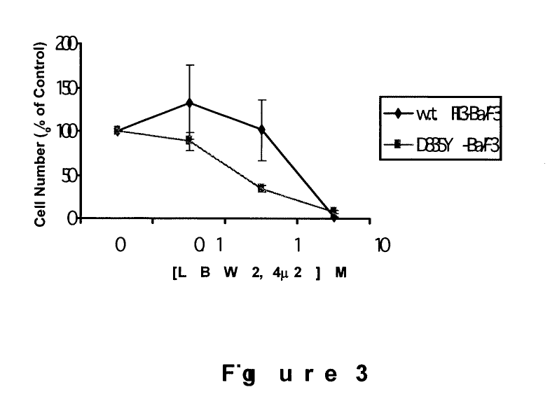

FIG. 3 illustrates three-day treatment of N-[1-cyclohexyl-2-oxo-2-(6-phenethyl-octahydro-pyrrolo[2,3-c]pyridin-1-yl-ethyl]-2-methylamino-propionamide on wild-type FLT3-Ba/F3 and D835Y-Ba/F3 cells with LBW242 (n=2).

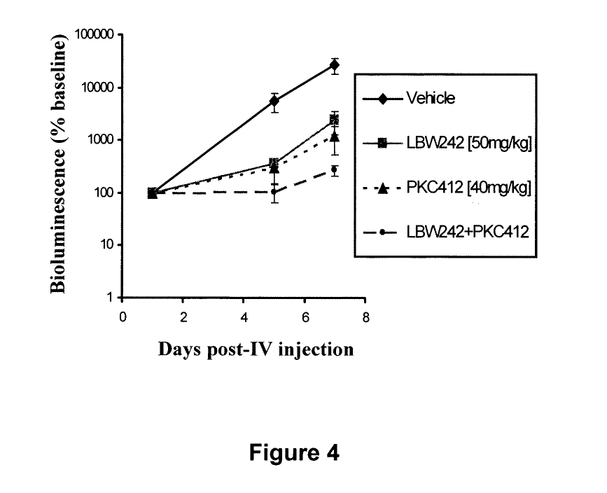

FIG. 4 illustrates NCR nude mice injected with 800,000 Ba/F3-FLT3-ITD-luc+ cells via IV tail vein, and then treated for up to 10 days by oral gavage with vehicle (NMP+PEG300), LBW242 (50 mg/kg).

### DETAILED DESCRIPTION OF THE INVENTION

Mediators of apoptotic signaling represent an attractive target for therapeutic intervention. “Second mitochondria-derived activator of caspase” (“Smac”) mediates apoptosis occurring through the intrinsic apoptotic pathway [see Du et al. (2000)], and binds to and inhibits the IAP family of proteins. See Liu et al. (2000) and Wu et al. (2000). Smac is likely the functional equivalent of Drosophila Reaper, Hid and Grim [see Vucic et al. (1998); McCarthy and Dixit (1998) and Goyal et al. (2000)]; the mouse Smac ortholog is DIABLO. See Verhagen et al. (2000). Identified human IAPs (c-IAP-1, c-IAP-2, and X-chromosome-linked IAP, or XIAP) bind procaspase-9 and prevent its activation. See Deveraux et al. (1998). IAPs also directly bind and inhibit active caspases [see Deveraux et al. (1997); Roy et al. (1997) and Deveraux et al. (1998)]; the BIR (“baculovirus IAP repeat”) domain is responsible for the anti-apoptotic activity of IAPs. See Takahashi et al. (1998). Members of the IAP protein family play a role in mediating apoptosis.

Examples of IAP inhibitors for use in the present invention include A compound according to formula (I):

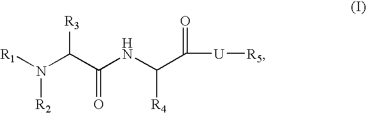

wherein
R₁ is H; C₁-C₄alkyl; C₁-C₄alkenyl; C₁-C₄ alkynyl or C₃-C₁₀cycloalkyl which are unsubstituted or substituted;
R₂ is H; C₁-C₄alkyl; C₁-C₄alkenyl; C₁-C₄alkynyl or C₃-C₁₀cycloalkyl which are unsubstituted or substituted;
R₃ is H; —CF₃; —C₂F₅; C₁-C₄alkyl; C₁-C₄alkenyl; C₁-C₄alkynyl; —CH₂—Z, or
R₂ and R₃, together with the nitrogen, form a het ring;
Z is H; —OH; F; Cl; —CH₃; —CF₃; —CH₂Cl; —CH₂F or —CH₂OH;
R₄ is C₁-C₁₆straight or branched alkyl; C₁-C₁₆alkenyl; C₁-C₁₆alkynyl; or —C₃-C₁₀cycloalkyl; —(CH₂)₁₋₆—Z₁; —(CH₂)₀₋₆-arylphenyl; and —(CH₂)₀₋₆-het; wherein alkyl, cycloalkyl and phenyl are unsubstituted or substituted;
Z₁ is —N(R₈)—C(O)—C₁-C₁₀alkyl; —N(R₈)—C(O)—(CH₂)₁₋₆—C₃-C₇cycloalkyl; —N(R₈)—C(O)—(CH₂)₀₋₆-phenyl; —N(R₈)—C(O)—(CH₂)₁₋₆-het; —C(O)—N(R₉)(R₁₀); —C(O)—O—C₁-C₁₀alkyl; —C(O)—O—(CH₂)₁₋₆—C₃-C₇cycloalkyl; —C(O)—O—(CH₂)₀₋₆-phenyl; —C(O)—O—(CH₂)₁₋₆-het; —O—C(O)—C₁-C₁₀alkyl; —O—C(O)—(CH₂)₁₋₆—C₃-C₇cycloalkyl; —O—C(O)—(CH₂)₀₋₆-phenyl; —O—C(O)—(CH₂)₁₋₆-het; wherein alkyl, cycloalkyl and phenyl are unsubstituted or substituted;
het is a 5- to 7-membered heterocyclic ring containing 1-4 heteroatoms selected from N, O and S, or an 8- to 12-membered fused ring system including at least one 5- to 7-membered heterocyclic ring containing 1, 2 or 3 heteroatoms selected from N, O and S, which heterocyclic ring or fused ring system is unsubstituted or substituted on a carbon or nitrogen atom;
R₈ is H; —CH₃; —CF₃; —CH₂OH or —CH₂Cl;
R₉ and R₁₀ are each independently H; C₁-C₄alkyl; C₃-C₇cycloalkyl; —(CH₂)₁₋₆—C₃-C₇cycloalkyl; —(CH₂)₀₋₆-phenyl; wherein alkyl, cycloalkyl and phenyl are unsubstituted or substituted, or
R₉ and R₁₀, together with the nitrogen, form het;
R₅ is H; C₁-C₁₀-alkyl; aryl; phenyl; C₃-C₇cycloalkyl; —(CH₂)₁₋₆—C₃-C₇cycloalkyl; —C₁-C₁₀alkyl-aryl; —(CH₂)₀₋₆—C₃-C₇cycloalkyl-(CH₂)₀₋₆-phenyl; —(CH₂)₀₋₄CH—((CH₂)₁₋₄-phenyl)₂; —(CH₂)₀₋₆—CH(phenyl)₂; -indanyl; —C(O)—C₁-C₁₀alkyl; —C(O)—(CH₂)₁₋₆—C₃-C₇-cycloalkyl; —C(O)—(CH₂)₀₋₆-phenyl; —(CH₂)₀₋₆—C(O)-phenyl; —(CH₂)₀₋₆-het; —C(O)—(CH₂)₁₋₆-het, or
R₅ is a residue of an amino acid, wherein the alkyl, cycloalkyl, phenyl and aryl substituents are unsubstituted or substituted;
U is a as shown in structure (II):

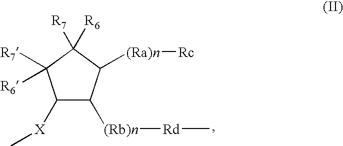

wherein
   n=0-5;
   X is —CH or N;
   Ra and Rb are independently an O, S, or N atom or C₀-C₈alkyl, wherein one or more of the carbon atoms in the alkyl chain may be replaced by a heteroatom selected from O, S or N, and where the alkyl may be unsubstituted or substituted;
   Rd is selected from:
      (a) —Re-Q-(Rf)ₚ(Rg)_{q}; or
      (b) Ar₁-D-Ar₂; or
      (c) Ar₁-D-Ar₂;
   Rc is H or Rc and Rd may together form a cycloalkyl or het; where if Rd and Rc form a cycloalkyl or het, R₆ is attached to the formed ring at a C or N atom;
      p and q are independently 0 or 1;
      Re is C₁-C₈alkyl or alkylidene, and Re which may be unsubstituted or substituted;
      Q is N, O, S, S(O) or S(O)₂;
      Ar₁ and Ar₂ are substituted or unsubstituted aryl or het;
      Rf and Rg are each independently none, or H; —C₁-C₁₀alkyl; C₁-C₁₀alkylaryl; —OH; —O—C₁-C₁₀alkyl; —(CH₂)₀₋₆—C₃-C₇cycloalkyl; —O—(CH₂)₀₋₆-aryl; phenyl; aryl; phenyl-phenyl; —(CH₂)₁₋₆-het; —O—(CH₂)₁₋₆-het; —OR₁₁; —C(O)—R₁₁; —C(O)—N(R₁₁)(R₁₂); —N(R₁₁)(R₁₂); —S—R₁₁; —S(O)—R₁₁; —S(O)₂—R₁₁; —S(O)₂—NR₁₁R₁₂; —NR₁₁—S(O)₂—R₁₂; S—C₁-C₁₀alkyl; aryl-C₁-C₄alkyl; het-C₁-C₄alkyl, wherein alkyl, cycloalkyl, het and aryl are unsubstituted or substituted; —SO₂—C₁-C₂alkyl; —SO₂—C₁-C₂alkylphenyl; —O—C₁-C₄alkyl, or
      R_{g} and R_{f} form a ring selected from het or aryl;
      D is —CO—; —C(O)— or C₁-C₇alkylene or arylene; —CF₂—; —O—; -or S(O)ₙᵣ where m is 0-2; 1,3dioaxolane; or C₁-C₇alkyl-OH; where alkyl, alkylene or arylene may be unsubstituted or substituted with one or more halogens, OH, —O—C₁-C₆alkyl, —S—C₁-C₆alkyl or —CF₃, or
      D is —N(Rh), wherein Rh is H; C₁-C₇alkyl (unsubstituted or substituted); aryl; —O(C₁-C₇cycloalkyl) (unsubstituted or substituted); C(O)—C₁₀-C₁₀alkyl; C(O)—C₀-C₁₀alkyl-aryl; C—O—C₁-C₁₀alkyl; C—O—C₀-C₁₀alkyl-aryl or SO₂—C₁₀-C₁₀-alkyl; SO₂—(C₀-C₁₀-alkylaryl);
   R₆, R₇, R′₆ and R′₇ are each independently H; —C₁-C₁₀alkyl; —C₁-C₁₀alkoxy; aryl-C₁-C₁₀alkoxy; —OH; —O—C₁-C₁₀alkyl; —(CH₂)₀₋₆—C₃-C₇cycloalkyl; —O—(CH₂)₀₋₆-aryl; phenyl; —(CH₂)₁₋₆-het; —O—(CH₂)₁₋₆-het; —OR₁₁; —C(O)—R₁₁; —C(O)—N(R₁₁)(R₁₂); —N(R₁₁)(R₁₂); —S—R₁₁; —S(O)—R₁₁; —S(O)₂—R₁₁; —S(O)₂—NR₁₁R₁₂, —NR₁₁—S(O)₂—R₁₂, wherein alkyl, cycloalkyl and aryl are unsubstituted or substituted; and R₆, R₇, R′₆ and R′₇ can be united to form a ring system;
   R₁₁ and R₁₂ are independently H; C₁-C₁₀alkyl; —(CH₂)₀₋₆—C₃-C₇cycloalkyl; —(CH₂)₀₋₆—(CH)₀₋₁(aryl)₁₋₂; —C(O)—C₁-C₁₀alkyl; —C(O)—(CH₂)₁₋₆—C₃-C₇cycloalkyl; —C(O)—O—(CH₂)₀₋₆-aryl; —C(O)—(CH₂)₀₋₆—O-fluorenyl; —C(O)—NH—(CH₂)₀₋₆-aryl; —C(O)—(CH₂)₀₋₆-aryl; —C(O)—(CH₂)₁₋₆-het; —C(S)—C₁-C₁₀alkyl; —C(S)—(CH₂)₁₋₆—C₃-C₇cycloalkyl; —C(S)—O—(CH₂)₀₋₆-aryl; —C(S)—(CH₂)₀₋₆—O-fluorenyl; —C(S)—NH—(CH₂)₀₋₆-aryl; —C(S)—(CH₂)₀₋₆-aryl; —C(S)—(CH₂)₁₋₆-het; wherein alkyl, cycloalkyl and aryl are unsubstituted or substituted, or
   R₁₁ and R₁₂ are a substituent that facilitates transport of the molecule across a cell membrane, or
   R₁₁ and R₁₂, together with the nitrogen atom, form het;
      wherein the alkyl substituents of R₁₁ and R₁₂ may be unsubstituted or substituted by one or more substituents selected from C₁-C₁₀alkyl, halogen, OH, —O—C₁-C₆alkyl, —S—C₁-C₆alkyl or —CF₃;
      substituted cycloalkyl substituents of R₁₁ and R₁₂ are substituted by one or more substituents selected from a C₁-C₁₀alkene; C₁-C₆alkyl; halogen; OH; —O—C₁-C₆alkyl; —S—C₁-C₆alkyl or —CF₃; and
      substituted phenyl or aryl of R₁₁ and R₁₂ are substituted by one or more substituents selected from halogen; hydroxy; C₁-C₄alkyl; C₁-C₄alkoxy; nitro; —CN; —O—C(O)—C₁-C₄alkyl and —C(O)—O—C₁-C₄-aryl,
         or pharmaceutically acceptable salts thereof.

Compounds within the scope of formula (I) and the process for their manufacture are disclosed in U.S. 60/835,000, which is hereby incorporated into the present application by reference. The preferred compounds are selected from the group consisting of:
(S)—N—((S)-1-cyclohexyl-2-{(S)-2-[4-(4-fluoro-benzoyl)-thiazol-2-yl]-pyrrolidin-1-yl}-2-oxo-ethyl)-2-methylamino-propionamide;
(S)—N—[(S)-cyclohexyl-(ethyl-{(S)-1-[5-(4-fluoro-benzoyl)-pyridin-3-yl]propyl}carbamoyl)-methyl]-2-methylamino-propionamide;
(S)—N—((S)-1-cyclohexyl-2-{(S)-2-[5-(4-fluoro-phenoxy)-pyridin-3-yl]-pyrrolidin-1-yl}-2-oxo-ethyl)-2-methylamino-propionamide; and
N-[1-cyclohexyl-2-(2-{2-[(4-fluorophenyl)-methyl-amino]-pyridin-4-yl}pyrrolidin-1-yl)-2-oxo-ethyl]-2-methylamino-propinamide;
   and pharmaceutically acceptable salts thereof.

In another embodiment, the IAP inhibitor is a compound of formula III:

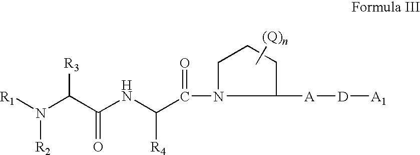

or pharmaceutically acceptable salts thereof, wherein

R₁ is H, C₁-C₄ alkyl, C₂-C₄ alkenyl, C₂-C₄ alkynyl or C₃-C₁₀ cycloalkyl, which R₁ may be unsubstituted or substituted;

R₂ is H, C₁-C₄ alkyl, C₂-C₄ alkenyl, C₂-C₄ alkynyl, C₃-C₁₀ cycloalkyl which R₂ may be unsubstituted or substituted;

R₃ is H, CF₃, C₂F₅, C₁-C₄ alkyl, C₂-C₄ alkenyl, C₂-C₄ alkynyl, CH₂—Z or R₂ and R₃ taken together with the nitrogen atom to which they are attached form a heterocyclic ring, which alkyl, alkenyl, alkynyl or het ring may be unsubstituted or substituted;

Z is H, OH, F, Cl, CH₃, CH₂Cl, CH₂F or CH₂OH;

R₄ is C₀₋₁₀ alkyl, C₃-C₁₀ cycloalkyl, wherein the C₀₋₁₀ alkyl, or cycloalkyl group is unsubstituted or substituted;

A is het, which may be substituted or unsubstituted;

D is C₁-C₇ alkylene or C₂-C₉ alkenylene, C(O), O, NR₇, S(O)r, C(O)—C₁-C₁₀ alkyl, O—C₁-C₁₀ alkyl, S(O)r—C₁-C₁₀ alkyl, C(O)C₀-C₁₀ arylalkyl OC₀-C₁₀ arylalkyl, or S(O)r C₀-C₁₀ arylalkyl, which alkyl and aryl groups may be unsubstituted or substituted;

r is 0, 1, or 2;

A₁ is a substituted aryl or unsubstituted or substituted het which substituents on aryl and het are halo, lower alkoxy, NR₅R₆, CN, NO₂ or SR₅;

each Q is independently H, C₁-C₁₀ alkyl, C₁-C₁₀ alkoxy, aryl C₁-C₁₀ alkoxy, OH, O—C₁-C₁₀-alkyl, (CH₂)₀₋₆—C₃-C₇ cycloalkyl, aryl, aryl C₁-C₁₀ alkyl, O—(CH₂)₀₋₆ aryl, (CH₂)₁₋₆het, het, O—(CH₂)₁₋₆-het, —OR₁₁, C(O)R₁₁, —C(O)N(R₁₁)(R₁₂), N(R₁₁)(R₁₂), SR₁₁, S(O)R₁₁, S(O)₂R₁₁, S(O)₂—N(R₁₁)(R₁₂), or NR₁₁—S(O)₂—(R₁₂), wherein alkyl, cycloalkyl and aryl are unsubstituted or substituted;

n is 0, 1, 2 or 3, 4, 5, 6 or 7;

het is a 5-7 membered monocyclic heterocyclic ring containing 1-4 heteroring atoms selected from N, O and S or an 8-12 membered fused ring system that includes one 5-7 membered monocyclic heterocyclic ring containing 1, 2, or 3 heteroring atoms selected from N, O and S, which het is unsubstituted or substituted;

R₁₁ and R₁₂ are independently H, C₁-C₁₀ alkyl, (CH₂)₀₋₆—C₃-C₇cycloalkyl, (CH₂)₀₋₆—(CH)₀₋₁(aryl)₁₋₂, C(O)—C₁-C₁₀alkyl, —C(O)—(CH₂)₁₋₆—C₃-C₇cycloalkyl, —C(O)—O—(CH₂)₀₋₆-aryl, —C(O)—(CH₂)₀₋₆—O-fluorenyl, C(O)—NH—(CH₂)₀₋₆-aryl, C(O)—(CH₂)₀₋₆-aryl, C(O)—(CH₂)₁₋₆-het, —C(S)—C₁-C₁₀alkyl, —C(S)—(CH₂)₁₋₆—C₃-C₇cycloalkyl, —C(S)—O—(CH₂)₀₋₆-aryl, —C(S)—(CH₂)₀₋₆—O-fluorenyl, C(S)—NH—(CH₂)₀₋₆-aryl, —C(S)—(CH₂)₀₋₆-aryl, C(S)—(CH₂)₁₋₆-het, C(O)R₁₁, C(O)NR₁₁R₁₂, C(O)OR₁₁, S(O)nR₁₁, S(O)ₘNR₁₁R₁₂, m=1 or 2, C(S)R₁₁, C(S)NR₁₁R₁₂, C(S)OR₁₁, wherein alkyl, cycloalkyl and aryl are unsubstituted or substituted; or R₁₁ and R₁₂ are a substituent that facilitates transport of the molecule across a cell membrane; or R₁₁ and R₁₂ together with the nitrogen atom form het;

wherein the alkyl substituents of R₁₁ and R₁₂ may be unsubstituted or substituted by one or more substituents selected from C₁-C₁₀alkyl, halogen, OH, O—C₁-C₆alkyl, —S—C₁-C₆alkyl, CF₃ or NR₁₁R₁₂;
substituted cycloalkyl substituents of R₁₁ and R₁₂ are substituted by one or more substituents selected from a C₂-C₁₀ alkene; C₁-C₆alkyl; halogen; OH; O—C₁-C₆alkyl; S—C₁-C₆alkyl, CF₃; or NR₁₁R₁₂ and
substituted het or substituted aryl of R₁₁ and R₁₂ are substituted by one or more substituents selected from halogen, hydroxy, C₁-C₄ alkyl, C₁-C₄ alkoxy, nitro, CN O—C(O)—C₁-C₄alkyl and C(O)—O—C₁-C₄-alkyl;

R₅, R₆ and R₇ are independently hydrogen, lower alkyl, aryl, aryl lower alkyl, cycloalkyl, or cycloalkyl lower alkyl, and

wherein the substituents on R₁, R₂, R₃, R₄, Q, and A and A₁ groups are independently halo, hydroxy, lower alkyl, lower alkenyl, lower alkynyl, lower alkanoyl, lower alkoxy, aryl, aryl lower alkyl, amino, amino lower alkyl, diloweralkylamino, lower alkanoyl, amino lower alkoxy, nitro, cyano, cyano lower alkyl, carboxy, lower carbalkoxy, lower alkanoyl, aryloyl, lower arylalkanoyl, carbamoyl, N-mono- or N,N-dilower alkyl carbamoyl, lower alkyl carbamic acid ester, amidino, guanidine, ureido, mercapto, sulfo, lower alkylthio, sulfoamino, sulfonamide, benzosulfonamide, sulfonate, sulfanyl lower alkyl, aryl sulfonamide, halogen substituted aryl sulfonate, lower alkylsulfinyl, arylsulfinyl; aryl-lower alkylsulfinyl, lower alkylarylsulfinyl, lower alkylsulfonyl, arylsulfonyl, aryl-lower alkylsulfonyl, lower aryl alkyl lower alkylarylsulfonyl, halogen-lower alkylmercapto, halogen-lower alkylsulfonyl, phosphono (—P(═O)(OH)₂), hydroxy-lower alkoxy phosphoryl or di-lower alkoxyphosphoryl, (R₉)NC(O)—NR₁₀R₁₃, lower alkyl carbamic acid ester or carbamates or —NR₈R₁₄, wherein R₈ and R₁₄ can be the same or different and are independently H or lower alkyl, or R₈ and R₁₄ together with the N atom form a 3- to 8-membered heterocyclic ring containing a nitrogen heteroring atoms and may optionally contain one or two additional heteroring atoms selected from nitrogen, oxygen and sulfur, which heterocyclic ring may be unsubstituted or substituted with lower alkyl, halo, lower alkenyl, lower alkynyl, hydroxy, lower alkoxy, nitro, amino, lower alkyl, amino, diloweralkyl amino, cyano, carboxy, lower carbalkoxy, formyl, lower alkanoyl, oxo, carbarmoyl, N-lower or N,N-dilower alkyl carbamoyl, mercapto, or lower alkylthio, and

R₉, R₁₀, and R₁₃ are independently hydrogen, lower alkyl, halogen substituted lower alkyl, aryl, aryl lower alkyl, halogen substituted aryl, halogen substituted aryl lower alkyl.

Some compounds which fall within compounds of formula III include: (S)—N—((S)-1-Cyclohexyl-2-{(S)-2-[4-(4-fluoro-benzoyl)-thiazol-2-yl]-pyrrolidin-1-yl}-2-oxo-ethyl)-2-methylamino-propionamide; (S)—N—[(S)-Cyclohexyl-(ethyl-{(S)-1-[5-(4-fluoro-benzoyl)-pyridin-3-yl]-propyl}carbamoyl)-methyl]-2-methylamino-propionamide; and (S)—N—((S)-1-Cyclohexyl-2-{(S)-2-[5-(4-fluoro-phenoxy)-pyridin-3-yl]-pyrrolidin-1-yl}-2-oxo-ethyl)-2-methylamino-propionamide; and pharmaceutically acceptable salts thereof.

These compounds of formula III are disclosed in PCT/US2007/074790 and U.S. Ser. No. 60/835,000; both herein incorporated by reference in their entirety.

Examples of other IAP inhibitors includes compounds disclosed in WO 05/097791 published on Oct. 20, 2005. A preferred compounds within the scope of formula (I) is N-[1-cyclohexyl-2-oxo-2-(6-phenethyl-octahydro-pyrrolo[2,3-c]pyridin-1-yl-ethyl]-2-methylamino-propionamide.

Additional IAP inhibitors include compounds disclosed in WO 04/005284, PCT/US2006/013984 and PCT/US2006/021850.

Other IAP inhibitor compounds for use in the present invention include those disclosed in WO 06/069063, WO 05/069888, US2006/0014700, WO 04/007529, US2006/0025347, WO 06/010118, WO 05/069894, WO 06/017295, WO 04/007529, and WO 05/094818.

In each case where citations of patent applications are given above, the subject matter relating to the compounds is hereby incorporated into the present application by reference. Comprised are likewise the pharmaceutically acceptable salts thereof, the corresponding racemates, diastereoisomers, enantiomers, tautomers, as well as the corresponding crystal modifications of above disclosed compounds where present, e.g., solvates, hydrates and polymorphs, which are disclosed therein. The compounds used as active ingredients in the combinations of the invention can be prepared and administered as described in the cited documents, respectively. Also within the scope of this invention is the combination of more than two separate active ingredients as set forth above, i.e., a pharmaceutical combination within the scope of this invention could include three active ingredients or more.

The terms “treatment” or “therapy” refer to the prophylactic or preferably therapeutic including, but not limited to, palliative, curing, symptom-alleviating, symptom-reducing, regulating and/or inhibiting treatment of said diseases, especially of the diseases mentioned below.

The term “AML”, as used herein, relates to an uncontrolled, quickly progressing growth of myeloid cells, e.g., granulocytes, as well as erythroid and megakaryotic cells and progenitors. In patients with AML the immature myeloid, erythroid or megakaryotic cells severely outnumber erythrocytes (red blood cells) leading to fatigue and bleeding, and also to increased susceptibility to infection. In children, as well as in adults, AML has a poor prognosis despite the use of aggressive chemotherapeutic protocols. Overall survival rates are 40-60%. Autologous bone marrow transplant preceded by myeloablative chemotherapy does not change the survival but an allogeneic bone marrow transplant preceded by aggressive chemotherapy might increase the survival rates up to 70%. Unfortunately, the availability of a matched sibling donor is limited. Therefore, new therapeutic strategies in AML treatment are necessary.

A warm-blooded animal (or patient) is preferably a mammal, especially a human.

The precise dosage of an IAP inhibitor compound to be employed depends upon several factors including the host, the nature and the severity of the condition being treated, the mode of administration. The IAP inhibitor compound can be administered by any route including orally, parenterally, e.g., intraperitoneally, intravenously, intramuscularly, subcutaneously, intratumorally, or rectally, or enterally. Preferably, the IAP inhibitor compound is administered orally, preferably at a daily dosage of 1-300 mg/kg body weight or, for most larger primates, a daily dosage of 50-5,000, preferably 500-3,000 mg. A preferred oral daily dosage is 1-75 mg/kg body weight or, for most larger primates, a daily dosage of 10-2,000 mg, administered as a single dose or divided into multiple doses, such as twice daily dosing.

Usually, a small dose is administered initially and the dosage is gradually increased until the optimal dosage for the host under treatment is determined. The upper limit of dosage is that imposed by side effects and can be determined by trial for the host being treated.

Dosage regimens must be titrated to the particular indication, the age, weight and general physical condition of the patient, and the response desired but generally doses will be from about 10 mg/day to about 500 mg/day as needed in single or multiple daily administration.

IAP inhibitor compounds may be combined with one or more pharmaceutically acceptable carriers and, optionally, one or more other conventional pharmaceutical adjuvants and administered enterally, e.g., orally, in the form of tablets, capsules, caplets, etc. or parenterally, e.g., intraperitoneally or intravenously, in the form of sterile injectable solutions or suspensions. The enteral and parenteral compositions may be prepared by conventional means.

N-[1-cyclohexyl-2-oxo-2-(6-phenethyl-octahydro-pyrrolo[2,3-c]pyridin-1-yl-ethyl]-2-methylamino-propionamide inhibits PKC412-sensitive and resistant mutant FLT3-expressing cells in vitro. N-[1-cyclohexyl-2-oxo-2-(6-phenethyl-octahydro-pyrrolo[2,3-c]pyridin-1-yl-ethyl]-2-methylamino-propionamide induces apoptosis, as measured via annexin-pi staining and caspase assays, was modestly observed with effective concentrations in the micromolar range.

N-[1-cyclohexyl-2-oxo-2-(6-phenethyl-octahydro-pyrrolo[2,3-c]pyridin-1-yl-ethyl]-2-methylamino-propionamide is effective against mutant FLT3 at doses that are physiologically achievable and well-tolerated in vivo.

### EXAMPLES

### Example 1

### Cell Lines and Cell Culture

The IL-3-dependent murine hematopoietic cell line Ba/F3 are transduced with either FLT3-ITD or FLT3-D835Y-containing MSCV retroviruses harboring a neomycin selectable marker, and selected for resistance to neomycin. See Kelly et al. (2002). FLT3-ITD transduced cells are selected for growth in G418 (1 mg/mL). PKC412-resistant Ba/F3 cell lines, which express FLT3-ITD harboring a mutation in the ATP-binding pocket (F691L, A627T, G697R, N676D), are developed as described previously. See Cools et al. (2004). The human AML-derived, FLT3-ITD-expressing cell line, MV4; 11 [see Quentmeier et al. (2003)], is provided by Dr. Scott Armstrong, Dana Farber Cancer Institute, Boston, Mass. The human AML-derived, FLT3-ITD-expressing cell line, MOLM-13, is modified to express luciferase and provided as MOLM13-luc+ by Dr. Andrew Kung, Dana Farber Cancer Institute, Boston, Mass. All cell lines are cultured with 5% CO₂ at 37° C., at a concentration of 2×10⁵ to 5×10⁵ in RPMI (Mediatech, Inc., Herndon, Va.) with 10% fetal calf serum and supplemented with 1% glutamine. Parental Ba/F3 cells expressing wild-type FLT3 are similarly cultured with 15% WEHI-conditioned medium as a source of IL-3. All transfected cell lines are cultured in media supplemented with 1 mg/mL G418.

### Example 2

### Chemical Compounds and Biologic Reagents

N-[1-cyclohexyl-2-oxo-2-(6-phenethyl-octahydro-pyrrolo[2,3-c]pyridin-1-yl-ethyl]-2-methylamino-propionamide is synthesized by Novartis Pharma AG, Basel, Switzerland, and dissolved in DMSO to make 10 mM stock solutions. Serial dilutions are then made, also in DMSO, to obtain final dilutions for cellular assays.

### Example 3

### Cell Viability and Apoptosis Analysis

The trypan blue exclusion assay has been previously described [see Weisberg et al. (2002)], and is used to determine proliferation of cells cultured in the presence and absence of LBW242. Cell viability is reported as percentage of control (untreated) cells. Error bars represent the standard error of the mean for each data point. Apoptosis of drug-treated cells is measured using the Annexin-V-Fluos Staining Kit (Boehringer Mannheim, Indianapolis, Ind.), as previously described. See Weisberg et al. (2002).

### Example 4

### Effects of N-[1-cyclohexyl-2-oxo-2-(6-phenethyl-octahydro-pyrrolo[2,3-c]pyridin-1-yl-ethyl]-2-methylamino-propionamide on Proliferation of PKC412-Sensitive and Resistant Mutant FLT3-Expressing Cells

N-[1-cyclohexyl-2-oxo-2-(6-phenethyl-octahydro-pyrrolo[2,3-c]pyridin-1-yl-ethyl]-2-methylamino-propionamide displays activity at relatively high doses (≧1 μM) against the PKC412-sensitive lines FLT3-ITD-Ba/F3 and D835Y-Ba/F3 cells, as well as the PKC412-resistant line G697R-Ba/F3 in culture, refer to FIGS. 1-3.

There are no inhibitory effects of N-[1-cyclohexyl-2-oxo-2-(6-phenethyl-octahydro-pyrrolo[2,3-c]pyridin-1-yl-ethyl]-2-methylamino-propionamide on cell growth of wild-type FLT3-expressing Ba/F3 cells at concentrations ≦1 μM; however, concentrations of N-[1-cyclohexyl-2-oxo-2-(6-phenethyl-octahydro-pyrrolo[2,3-c]pyridin-1-yl-ethyl]-2-methylamino-propionamide >1 μM led to death of these cells (FIG. 3).

For the FLT3-ITD-Ba/F3 and G697R-Ba/F3 lines, induction of apoptosis and caspase activity following 2 days and 3 days, respectively, of culturing in the presence of 1 μM N-[1-cyclohexyl-2-oxo-2-(6-phenethyl-octahydro-pyrrolo[2,3-c]pyridin-1-yl-ethyl]-2-methylamino-propionamide are found. Mutant FLT3-expressing cells are treated for 2 days in parallel with either 1 μM N-[1-cyclohexyl-2-oxo-2-(6-phenethyl-octahydro-pyrrolo[2,3-c]pyridin-1-yl-ethyl]-2-methylamino-propionamide in the presence and absence of WEHI (used as a source of IL-3). In contrast to PKC412-treated cells, which are fully rescued from the cytotoxic effects of PKC412 by WEHI, supplementation of culture media with WEHI did not rescue N-[1-cyclohexyl-2-oxo-2-(6-phenethyl-octahydro-pyrrolo[2,3-c]pyridin-1-yl-ethyl]-2-methylamino-propionamide-treated cells, showing that N-[1-cyclohexyl-2-oxo-2-(6-phenethyl-octahydro-pyrrolo[2,3-c]pyridin-1-yl-ethyl]-2-methylamino-propionamide, consistent with its proposed mechanisms of inhibition of IAP, does not selectively inhibit mutant FLT3, but interferes with viability.

### Example 5

### In Vivo Investigation of Effects of N-[1-cyclohexyl-2-oxo-2-(6-phenethyl-octahydro-pyrrolo[2,3-c]pyridin-1-yl-ethyl]-2-methylamino-propionamide

To directly assess the in vivo anti-tumor efficacy of N-[1-cyclohexyl-2-oxo-2-(6-phenethyl-octahydro-pyrrolo[2,3-c]pyridin-1-yl-ethyl]-2-methylamino-propionamide, a mouse model of acute leukemia in which tumor burden is quantified by non-invasive imaging of luminescent tumor cells (FIG. 4). Murine FLT3-ITD-Ba/F3 cells are engineered to stably express firefly luciferase, and NCr nude mice are then inoculated with these cells. Non-invasive imaging is used to serially assess tumor burden, and mice with established leukemia are divided into cohorts with similar tumor burden. N-[1-cyclohexyl-2-oxo-2-(6-phenethyl-octahydro-pyrrolo[2,3-c]pyridin-1-yl-ethyl]-2-methylamino-propionamide is administered via oral gavage, as was vehicle.

Mice are given vehicle alone, N-[1-cyclohexyl-2-oxo-2-(6-phenethyl-octahydro-pyrrolo[2,3-c]pyridin-1-yl-ethyl]-2-methylamino-propionamide (50 mg/kg) (FIG. 4). The lowest tumor burden as assessed by bioluminescence is observed to be in the drug combination group on days 5 and 7 post-IV injection of FLT3-1TD-Ba/F3-luc+ cells (and corresponding to 4 and 6 days of drug treatment, respectively). The Student t-test is used for statistical evaluation of bioluminescence results as observed on day 7 post-IV injection: p≦0.056247 (vehicle versus N-[1-cyclohexyl-2-oxo-2-(6-phenethyl-octahydro-pyrrolo[2,3-c]pyridin-1-yl-ethyl]-2-methylamino-propionamide alone). Statistical evaluation (via Student t-test) for day 5 post-IV injection yielded: p≦0.077299 (vehicle versus N-[1-cyclohexyl-2-oxo-2-(6-phenethyl-octahydro-pyrrolo[2,3-c]pyridin-1-yl-ethyl]-2-methylamino-propionamide alone).

### Example 6

### Single Agent Activity of (S)—N—((S)-1-Cyclohexyl-2-{(S)-2-[4-(4-fluoro-benzoyl)-thiazol-2-yl]-pyrrolidin-1-yl}-2-oxo-ethyl)-2-methylamino-propionamide in Hematological Cell Lines

The EC50s were determined using CellTiter-Glo (Promega), a bioluminescent, cell viability assay measuring ATP levels in viable cells. Cells were plated in assay plates and incubated with a range of compound concentrations for 72 hours. Cells were lysed and ATP levels were determined using CellTiter-Glo reagent on a luminometer according to manufacturer's instructions. EC50 refers to the concentration of compound that inhibited 50% of cell growth.

TABLE 1

| Cell line | Disease of patient | EC50 (uM) |
|---|---|---|
| NB-4 | AML | 0.0146 |
| ML-2 | AML | 0.015 |
| Reh | B-ALL | 0.0791 |
| LAMA-84 | CML | 0.203 |
| HSB-2 | T-ALL | 0.4 |
| CCRF-CEM | T-ALL | 0.676 |
| EM-2 | CML | 0.696 |
| EOL-1 | AML | 1.657 |
| KU812 | CML | 1.942 |
| JK-1 | CML | 3.493 |
| NALM-1 | CML | 3.545 |
| PL-21 | AML | 3.549 |
| CEM/C2 | T-ALL | 3.831 |
| OCI-AML3 | AML | 4.077 |
| NOMO-1 | AML | 4.077 |
| CEM/C1 | T-ALL | 4.6 |
| KE-37 | T-ALL | 5.1 |
| CCRF-HSB-2 | T-ALL | 6.3 |
| L-428 | HL | 7.3 |
| D1.1 | T-ALL | 7.349 |
| Kasumi-1 | AML | 8.641 |
| RL | B-NHL | 9.679 |
| MOLT-3 | T-ALL | 10.186 |
| KARPAS-45 | T-ALL | 10.5 |
| Karpas-299 | ALCL | 10.825 |
| SR | LCIL | 11.632 |
| JURL-MK1 | CML | 11.768 |
| JURL-MK2 | CML | 12.743 |
| EM-3 | CML | 13.788 |
| MV-4-11 | AML | 14.032 |

What is claimed is:

1. A method of treating a warm-blooded animal having acute myeloid leukemia (AML), comprising administering to said animal a therapeutically effective amount of a compound according to formula (III): 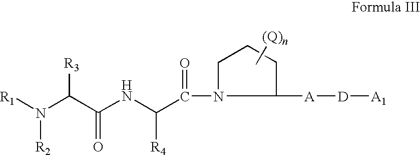
or pharmaceutically acceptable salts thereof, wherein
R₁ is H, C₁-C₄ alkyl, C₂-C₄ alkenyl, C₂-C₄ alkynyl or C₃-C₁₀ cycloalkyl, which R₁ may be unsubstituted or substituted;
R₂ is H, C₁-C₄ alkyl, C₂-C₄ alkenyl, C₂-C₄ alkynyl, C₃-C₁₀ cycloalkyl which R₂ may be unsubstituted or substituted;
R₃ is H, CF₃, C₂F₅, C₁-C₄ alkyl, C₂-C₄ alkenyl, C₂-C₄ alkynyl, CH₂—Z or R₂ and R₃ taken together with the nitrogen atom to which they are attached form a heterocyclic ring, which alkyl, alkenyl, alkynyl or het ring may be unsubstituted or substituted;
Z is H, OH, F, Cl, CH₃, CH₂Cl, CH₂F or CH₂OH;
R₄ is C₀₋₁₀ alkyl, C₃-C₁₀ cycloalkyl, wherein the C₀₋₁₀ alkyl, or cycloalkyl group is unsubstituted or substituted;
A is het, which may be substituted or unsubstituted;
D is C₁-C₇ alkylene or C₂-C₉ alkenylene, C(O), O, NR₇, S(O)r, C(O)—C₁-C₁₀ alkyl, O—C₁-C₁₀ alkyl, S(O)r-C₁-C₁₀ alkyl, C(O)C₀-C₁₀ arylalkyl OC₀-C₁₀ arylalkyl, or S(O)r C₀-C₁₀ arylalkyl, which alkyl and aryl groups may be unsubstituted or substituted;
r is 0, 1, or 2;
A₁ is a substituted aryl or unsubstituted or substituted het which substituents on aryl and het are halo, lower alkoxy, NR₅R₆, CN, NO₂ or SR₅;
each Q is independently H, C₁-C₁₀ alkyl, C₁-C₁₀ alkoxy, aryl C₁-C₁₀ alkoxy, OH, O—C₁-C₁₀-alkyl, (CH₂)₀₋₆—C₃-C₇ cycloalkyl, aryl, aryl C₁-C₁₀ alkyl, O—(CH₂)₀₋₆ aryl, (CH₂)₁₋₆het, het, O—(CH₂)₁₋₆het, —OR₁₁, C(O)R₁₁, —C(O)N(R₁₁)(R₁₂), N(R₁₁)(R₁₂), SR₁₁, S(O)R₁₁, S(O)₂R₁₁, S(O)₂—N(R₁₁)(R₁₂), or NR₁₁—S(O)₂—(R₁₂), wherein alkyl, cycloalkyl and aryl are unsubstituted or substituted;
n is 0, 1, 2 or 3, 4, 5, 6 or 7;
het is a 5-7 membered monocyclic heterocyclic ring containing 1-4 heteroring atoms selected from N, O and S or an 8-12 membered fused ring system that includes one 5-7 membered monocyclic heterocyclic ring containing 1, 2, or 3 heteroring atoms selected from N, O and S, which het is unsubstituted or substituted;
R₁₁ and R₁₂ are independently H, C₁-C₁₀ alkyl, (CH₂)₀₋₆—C₃-C₇cycloalkyl, (CH₂)₀₋₆—(CH)₀₋₁(aryl)₁₋₂, C(O)—C₁-C₁₀alkyl, —C(O)—(CH₂)₁₋₆—C₃-C₇cycloalkyl, —C(O)—O—(CH₂)₀₋₆-aryl, —C(O)—(CH₂)₀₋₆—O-fluorenyl, C(O)—NH—(CH₂)₀₋₆-aryl, C(O)—(CH₂)₀₋₆-aryl, C(O)—(CH₂)₁₋₆-het, —C(S)—C₁-C₁₀alkyl, —C(S)—(CH₂)₁₋₆—C₃-C₇cycloalkyl, —C(S)—O—(CH₂)₀₋₆-aryl, —C(S)—(CH₂)₀₋₆—O-fluorenyl, C(S)—NH—(CH₂)₀₋₆-aryl, —C(S)—(CH₂)₀₋₆-aryl or C(S)—(CH₂)₁₋₆-het, C(O)R₁₁, C(O)NR₁₁R₁₂, C(O)OR₁₁, S(O)nR₁₁, S(O)ₘNR₁₁R₁₂, m=1 or 2, C(S)R₁₁, C(S)NR₁₁R₁₂, C(S)OR₁₁, wherein alkyl, cycloalkyl and aryl are unsubstituted or substituted; or R₁₁ and R₁₂ are a substituent that facilitates transport of the molecule across a cell membrane; or R₁₁ and R₁₂ together with the nitrogen atom form het;
wherein the alkyl substituents of R₁₁ and R₁₂ may be unsubstituted or substituted by one or more substituents selected from C₁-C₁₀alkyl, halogen, OH, O—C₁-C₆alkyl, —S—C₁-C₆alkyl, CF₃ or NR₁₁R₁₂;
substituted cycloalkyl substituents of R₁₁ and R₁₂ are substituted by one or more substituents selected from a C₂-C₁₀ alkene; C₁-C₆alkyl; halogen; OH; O—C₁-C₆alkyl; S—C₁-C₆alkyl, CF₃; or NR₁₁R₁₂ and
substituted het or substituted aryl of R₁₁ and R₁₂ are substituted by one or more substituents selected from halogen, hydroxy, C₁-C₄ alkyl, C₁-C₄ alkoxy, nitro, CN O—C(O)—C₁-C₄alkyl and C(O)—O—C₁-C₄-alkyl;
R₅, R₆ and R₇ are independently hydrogen, lower alkyl, aryl, aryl lower alkyl, cycloalkyl, or cycloalkyl lower alkyl, and
wherein the substituents on R₁, R₂, R₃, R₄, Q, and A and A₁ groups are independently halo, hydroxy, lower alkyl, lower alkenyl, lower alkynyl, lower alkanoyl, lower alkoxy, aryl, aryl lower alkyl, amino, amino lower alkyl, diloweralkylamino, lower alkanoyl, amino lower alkoxy, nitro, cyano, cyano lower alkyl, carboxy, lower carbalkoxy, lower alkanoyl, aryloyl, lower arylalkanoyl, carbamoyl, N-mono- or N,N-dilower alkyl carbamoyl, lower alkyl carbamic acid ester, amidino, guanidine, ureido, mercapto, sulfo, lower alkylthio, sulfoamino, sulfonamide, benzosulfonamide, sulfonate, sulfanyl lower alkyl, aryl sulfonamide, halogen substituted aryl sulfonate, lower alkylsulfinyl, arylsulfinyl; aryl-lower alkylsulfinyl, lower alkylarylsulfinyl, lower alkylsulfonyl, arylsulfonyl, aryl-lower alkylsulfonyl, lower aryl alkyl lower alkylarylsulfonyl, halogen-lower alkylmercapto, halogen-lower alkylsulfonyl, phosphono(—P(═O)(OH)₂), hydroxy-lower alkoxy phosphoryl or di-lower alkoxyphosphoryl, (R₉)NC(O)—NR₁₀R₁₃, lower alkyl carbamic acid ester or carbamates or —NR₈R₁₄, wherein R₈ and R₁₄ can be the same or different and are independently H or lower alkyl, or R₈ and R₁₄ together with the N atom form a 3- to 8-membered heterocyclic ring containing a nitrogen heteroring atoms and may optionally contain one or two additional heteroring atoms selected from nitrogen, oxygen and sulfur, which heterocyclic ring may be unsubstituted or substituted with lower alkyl, halo, lower alkenyl, lower alkynyl, hydroxy, lower alkoxy, nitro, amino, lower alkyl, amino, diloweralkyl amino, cyano, carboxy, lower carbalkoxy, formyl, lower alkanoyl, oxo, carbarmoyl, N-lower or N,N-dilower alkyl carbamoyl, mercapto, or lower alkylthio, and
R₉, R₁₀, and R₁₃ are independently hydrogen, lower alkyl, halogen substituted lower alkyl, aryl, aryl lower alkyl, halogen substituted aryl, halogen substituted aryl lower alkyl.

2. A method according to claim 1, wherein the AML is resistant to conventional chemotherapy.

3. A method according to claim 1, wherein the warm-blooded animal is a human.

4. A method according to claim 3, wherein the human is a juvenile human.

5. A pharmaceutical composition comprising a compound of formula (III), as defined in claim 1, optionally together with a pharmaceutical carrier.

6. Use of a pharmaceutical composition according to claim 5 for the treatment of AML.

7. A commercial package comprising a compound of formula (III) useful in AML treatment as defined in claim 1, together with instructions for simultaneous, separate or sequential use thereof in the treatment of AML.

8. The use of a compound of formula (II), as defined in claim 1, for the preparation of a medicament for the treatment of AML.
